(19) United States
(12) Patent Application Publication
(10) Pub. No.: US 2026/0193610 A1
(43) Pub. Date: Jul. 9, 2026

(54) METHOD FOR DIFFERENTIATING PLATELETS, CULTURE MEDIUM, AND USE

(71) Applicant: HEMACELL BIOTECHNOLOGY INC., Suzhou, Jiangsu (CN)

(72) Inventors: Fangfang ZHU, Suzhou, Jiangsu (CN); Hao ZHU, Suzhou, Jiangsu (CN)

(21) Appl. No.: 19/132,019

(22) PCT Filed: Dec. 4, 2023
(86) PCT No.: PCT/CN2023/136060

§ 371 Date: May 22, 2025

(30) Foreign Application Priority Data
Dec. 6, 2022 (WO) PCT/CN2022/136731

(51) Int. Cl. C12N 5/078

(52) U.S. Cl. CPC C12N 5/0644; C12N 2500/02; C12N 2500/32; C12N 2500/38; C12N 2501/125; C12N 2501/145; C12N 2501/2303; C12N 2501/2306; C12N 2501/26; C12N 2502/115

(57) ABSTRACT

Provided is a method for differentiating platelet cells, comprising adding a human platelet lysate (hPL) to a platelet differentiation culture medium. Further provided are a culture medium for differentiating platelets and use thereof.

### TECHNICAL FIELD

The present application relates to the field of biomedicine, and specifically to a method for differentiating platelets.

### BACKGROUND ART

Platelets are small circulating anucleated cells that are differentiated from mature megakaryocytes and contribute to hemostasis by aggregating and forming emboli in vascular injury. The life span of a single platelet in the body is about 7-10 days, so the human body produces a large number of fresh platelets every day to maintain a normal platelet count. As an inherent component of organisms, platelets may escape clearance by the immune system and are closely related to physiological processes such as vascular endothelial damage repair, immune response, atherosclerosis formation, neurodegeneration, tumor growth and metastasis, and therefore have broad application prospects.

Platelet transfusion is a life-saving method used to prevent bleeding or stop ongoing bleeding in patients suffering from thrombocytopenia and functional platelet disorders. Furthermore, it may be used to prevent bleeding in patients having thrombocytopenia caused by chemotherapy for malignant tumors or hematopoietic stem cell transplantation. Currently, platelets mainly come from donations from donors. Due to the short storage time of donor-derived platelets and the insufficient number of donors, platelets have become a scarce resource. At the same time, there is a risk of bacterial contamination and deterioration in the process of platelet processing, preparation and transfusion. Therefore, it is very necessary to develop alternative strategies.

Human platelet lysate (hPL) is widely used in the research, development and production of biopharmaceuticals for cell therapies. Many studies have confirmed that its use as a cell culture additive in the culture of human mesenchymal stem cells (MSCs) can better maintain cell activity, promote cell proliferation, and delay cell senescence caused by subculturing, which is more advantageous than serum-free culturing and culturing cells supplemented with fetal bovine serum. However, there is no comprehensive and detailed study on the use of the human platelet lysate in the cultures for inducing differentiation into platelets in vitro.

In recent years, the demand for platelets has been increasing day by day. The current methods for inducing the production of platelets in vitro are relatively complex and have low efficiency and yield. Therefore, it is urgent to develop methods that can effectively differentiate and/or proliferate and/or maintain platelets, and use the methods to produce platelets for treating diseases.

### SUMMARY OF THE INVENTION

The present application provides a method for differentiating platelets. The method provided in the present application can effectively improve the proliferation and/or differentiation efficiency of platelets by adding a human platelet lysate (hPL) which contains abundant cytokines and nutrients to a platelet differentiation culture medium, while facilitating the maintenance of platelet activity.

In one aspect, the present application provides a method for inducing the proliferation of pluripotent cells and/or their differentiation into platelets, comprising adding a human platelet lysate (hPL) to a platelet differentiation culture medium.

In one aspect, the present application provides a method for inducing the proliferation of hematopoietic stem/progenitor cells and/or their differentiation into platelets, comprising adding a human platelet lysate (hPL) to a platelet differentiation culture medium.

In one aspect, the present application provides a method for inducing the proliferation of megakaryocytic progenitor cells (MKPs) and/or their differentiation into platelets, comprising adding a human platelet lysate (hPL) to a platelet differentiation culture medium.

In some embodiments, the pluripotent cells in the method are derived from mammals.

In some embodiments, the pluripotent cells in the method are human embryonic stem cells.

In some embodiments, the pluripotent cells in the method are human induced pluripotent stem cells.

In some embodiments, the hematopoietic stem/progenitor cells in the method are derived from human embryonic stem cells.

In some embodiments, the hematopoietic stem/progenitor cells in the method are derived from human induced pluripotent stem cells.

In some embodiments, the hematopoietic stem/progenitor cells in the method are derived from human blood ex vivo.

In some embodiments, the hematopoietic stem/progenitor cells in the method are derived from umbilical cord blood.

In some embodiments, the hematopoietic stem/progenitor cells in the method are derived from bone marrow.

In some embodiments, the hematopoietic stem/progenitor cells in the method are CD34+ hematopoietic stem/progenitor cells.

In some embodiments, the MKPs in the method are derived from human embryonic stem cells.

In some embodiments, the MKPs in the method are derived from human induced pluripotent stem cells.

In some embodiments, the MKPs in the method are derived from human blood ex vivo.

In some embodiments, the MKPs in the method are derived from umbilical cord blood.

In some embodiments, the MKPs in the method are derived from bone marrow.

In some embodiments, the MKPs in the method are derived from hematopoietic stem/progenitor cells.

In some embodiments, the MKPs in the method are derived from CD34+ hematopoietic stem/progenitor cells.

In some embodiments, the platelet differentiation culture medium in the method is free of serum.

In some embodiments, the platelet differentiation culture medium in the method comprises a basal culture medium, the basal culture medium is selected from one or more in the group of IMDM, MEM, Ham's F12, mTeSR1, APEL, StemSpan™ SFEM II, DMEM, and RPMI1640. In some embodiments, the basal culture medium comprises one or more of StemSpan™ SFEM II and IMDM. In some embodiments, the basal culture medium is StemSpan™ SFEM II. In some embodiments, the basal culture medium is IMDM.

In some embodiments, the platelet differentiation culture medium in the method further comprises nutrients, extracts, growth factors, hormones, cytokines, and culture medium supplements.

In some embodiments, the platelet differentiation culture medium in the method further comprises one or more of IL-3, IL-6, SCF (stem cell factor), TPO (thrombopoietin), FLt3L (Fms-related tyrosine kinase 3 ligand), N2 supplement, B27 supplement, NEAAs (non-essential amino acids), Glutamax (glutamine), ITS (insulin, transferrin, selenium), and Ascorbic acid.

In some embodiments, the platelet differentiation culture medium in the method further comprises IL-3, IL-6, SCF, TPO, and FLt3L.

In some embodiments, the platelet differentiation culture medium in the method further comprises IL-6, SCF, TPO, N2 supplement, B27 supplement, NEAAs, Glutamax, ITS, and Ascorbic acid.

In some embodiments, the platelet differentiation culture medium in the method further comprises platelet differentiation culture medium I, the platelet differentiation culture medium I comprises StemSpan™ SFEM II, IL-3, IL-6, SCF, TPO, FLt3L, and hPL.

In some embodiments, the platelet differentiation culture medium in the method further comprises platelet differentiation culture medium II, the platelet differentiation culture medium II comprises IMDM, N2 supplement, B27 supplement, NEAAs, Glutamax, ITS, Ascorbic acid, IL-6, SCF, TPO, and hPL.

In some embodiments, the method comprises culturing the hematopoietic stem/progenitor cells in the platelet differentiation culture medium I for about 7 days.

In some embodiments, the method comprises culturing the MKPs in the platelet differentiation culture medium II for about 10 days.

In some embodiments, the method comprises continuously culturing the MKPs in the platelet differentiation culture medium II supplemented with a metalloproteinase inhibitor for about 2 days.

In some embodiments, the method comprises culturing cells under a condition of 35-39° C.

In some embodiments, the method comprises culturing cells under a condition of about 3%-7% CO₂.

In another aspect, the present application further provides a culture medium comprising the platelet differentiation culture medium supplemented with a human platelet lysate (hPL).

In some embodiments, the platelet differentiation culture medium in the culture medium is free of serum.

In some embodiments, the platelet differentiation culture medium in the culture medium comprises a basal culture medium.

In some embodiments, the basal culture medium in the culture medium is selected from one or more in the group of IMDM, MEM, Ham's F12, mTeSR1, APEL, StemSpan™ SFEM II, DMEM, and RPMI1640. In some embodiments, the basal culture medium comprises one or more of StemSpan™ SFEM II and IMDM. In some embodiments, the basal culture medium is StemSpan™ SFEM II. In some embodiments, the basal culture medium is IMDM.

In some embodiments, the platelet differentiation culture medium in the culture medium further comprises nutrients, extracts, growth factors, hormones, cytokines, and culture medium supplements.

In some embodiments, the platelet differentiation culture medium in the culture medium further comprises one or more of IL-3, IL-6, SCF, TPO, FLt3L, N2 supplement, B27 supplement, NEAAs, Glutamax, ITS, and Ascorbic acid.

In some embodiments, the platelet differentiation culture medium in the culture medium further comprises IL-3, IL-6, SCF, TPO, and FLt3L.

In some embodiments, the platelet differentiation culture medium in the culture medium further comprises IL-6, SCF, TPO, N2 supplement, B27 supplement, NEAAs, Glutamax, ITS, and Ascorbic acid.

In some embodiments, the platelet differentiation culture medium in the culture medium further comprises platelet differentiation culture medium I, the platelet differentiation culture medium I comprises StemSpan™ SFEM II, IL-3, IL-6, SCF, TPO, FLt3L, and hPL.

In some embodiments, the platelet differentiation culture medium in the culture medium further comprises platelet differentiation culture medium II, the platelet differentiation culture medium II comprises IMDM, N2 supplement, B27 supplement, NEAAs, Glutamax, ITS, Ascorbic acid, IL-6, SCF, TPO, and hPL.

In some embodiments, the concentration of the IL-3 in the method and the culture medium is about 5-100 ng/mL.

In some embodiments, the concentration of the IL-6 in the method and the culture medium is about 5-100 ng/mL.

In some embodiments, the concentration of the SCF in the method and the culture medium is about 5-100 ng/mL.

In some embodiments, the concentration of the TPO in the method and the culture medium is about 5-100 ng/mL.

In some embodiments, the concentration of the FLt3L in the method and the culture medium is about 5-100 ng/mL.

In some embodiments, the concentration of the N2 supplement in the method and the culture medium is about 0.25%-1%.

In some embodiments, the concentration of the B27 supplement in the method and the culture medium is about 0.5%-2%.

In some embodiments, the concentration of the Glutamax in the method and the culture medium is about 0.5%-2%.

In some embodiments, the concentration of the ITS in the method and the culture medium is about 0.5%-2%.

In some embodiments, the concentration of the Ascorbic acid in the method and the culture medium is about 50-200 μM.

In some embodiments, the concentration of the hPL in the method and the culture medium is about 0.5%-10%.

In another aspect, the present application further provides a use of the method in differentiating platelets.

In another aspect, the present application further provides a use of the culture medium in inducing the proliferation of pluripotent cells and/or their differentiation into platelets.

In another aspect, the present application further provides a use of the culture medium in inducing the proliferation of hematopoietic stem/progenitor cells and/or their differentiation into platelets.

In another aspect, the present application further provides a use of the culture medium in inducing the proliferation of MKPs and/or their differentiation into platelets.

In another aspect, the present application further provides a composition comprising pluripotent cells and the culture medium.

In another aspect, the present application further provides a composition comprising hematopoietic stem/progenitor cells and the culture medium.

In another aspect, the present application further provides a composition comprising MKPs and the culture medium.

In another aspect, the present application further provides a culture platform for obtaining platelets, which comprises the method and the culture medium.

In another aspect, the present application further provides a method for preventing and/or treating diseases, which comprises administering the platelets to a subject in need thereof, wherein the platelets are obtained by using the method, the culture medium, the composition or the culture platform.

Those skilled in the art may easily discern other aspects and advantages of the present application from the following detailed description. Only exemplary embodiments of the present application are shown and described in the following detailed description. As those skilled in the art will realize, the contents of the present application enable those skilled in the art to make modifications to the specific embodiments disclosed without departing from the spirit and scope of the invention covered by the present application. Accordingly, the descriptions in the specification of the present application are illustrative only and not limited.

### BRIEF DESCRIPTION OF DRAWINGS

The specific features of the invention to which the present application relates are set forth in the appended claims. The features and advantages of the invention to which the present application relates may be better understood by reference to the exemplary embodiments described in detail below and the drawings. A brief description of the drawings is as follows:

FIG. 1 shows the effects of culturing cells in a culture medium with and without hPL as described in the present application on cell proliferation.

FIG. 2 shows the detection of the cultured cells on Day 7 as described in the present application.

FIG. 3 shows the detection of the cultured cells on Day 14 as described in the present application.

### DETAILED DESCRIPTION

The embodiments of the invention of the present application will be illustrated below with specific examples. Those skilled in the art may easily understand other advantages and effects of the invention of the present application from the disclosure of the specification.

### DEFINITION OF TERMS

In the present application, the term “pluripotent cell” generally refers to a cell having the potential for proliferation and differentiation. Pluripotent cells may be functionally defined as cells that: (1) have the ability to differentiate into different cell types and, in some cases, generate only one specialized cell type, and (2) are capable of making long-term self-renewal, generating one or more cells that may be the same as or different from the original cell type. The source and preparation method of pluripotent cells are not limited. For example, the pluripotent cells may be naturally obtained or artificially modified. For example, the pluripotent cells may include embryonic stem cells, induced pluripotent stem cells, and the like.

In the present application, the term “hematopoietic stem/progenitor cells” generally refers to cells that have the ability of long-term self-renewal and the potential to differentiate into various types of mature blood cells. The source and preparation method of hematopoietic stem cells are not limited. For example, the hematopoietic stem cells can be differentiated from pluripotent cells or can be separated from bone marrow or blood. Hematopoietic stem cells can differentiate into many types of cells, for example, bone marrow lineage cells (e.g., monocytes and macrophages, neutrophils, basophils, eosinophils, red blood cells, megakaryocytes/platelets, dendritic cells), and lymphoid lineage cells (e.g., T-cells, B-cells, NK-cells).

In the present application, the term “megakaryocytic progenitor cell” is also called “megakaryocyte”, which generally refers to a cell that can produce platelets. Megakaryocytic progenitor cells are characterized by large polyploid nuclei, large cell size, and rich cytoplasm, allowing each cell to produce thousands of platelets. The source and preparation method of megakaryocytic progenitor cells are not limited. For example, the megakaryocytic progenitor cells can be differentiated from pluripotent cells or can be isolated in vivo.

In the present application, the term “human embryonic stem cell”, also referred to as “embryonic stem cell”, abbreviated as “ESC”, generally refers to cells characterized by unlimited proliferation, self-renewal and multidirectional differentiation. Embryonic stem cells are stem cells obtained from the undifferentiated inner cell mass of the blastocyst (early embryonic stage). There is no restriction on their source and preparation method. Embryonic stem cells may be induced to differentiate into almost all cell types of the organism, whether in vitro or in vivo. For example, the cell types may be hematopoietic stem cells, nerve cells, cardiomyocytes, etc.

In the present application, the term “human induced pluripotent stem cells” may be abbreviated as iPS cells or iPSCs, and generally refers to a class of pluripotent stem cells prepared by an artificial means from non-pluripotent cells. Induced pluripotent stem cells can be obtained by introducing specific transcription factors to reprogram terminally differentiated somatic cells. For example, the terminally differentiated somatic cells can be fibroblasts, hematopoietic stem cells, muscle cells, neurons, epidermal cells and the like.

In the present application, the term “human platelet lysate (hPL)” is generally derived from human platelets, which comprises a variety of cell growth factors. In the present application, the human platelet lysate can comprise platelets from various sources. For example, the human platelet lysate can be derived from human platelets collected from blood donation. For example, the human platelet lysate can be derived from the platelets isolated and purified from blood samples. For example, the human platelet lysate can be derived from the platelets obtained by differentiation from various cells, e.g., the platelets obtained by differentiation from hematopoietic stem cells, e.g., the platelets obtained by differentiation from induced pluripotent stem cells (iPSCs), e.g., the platelets obtained by differentiation from megakaryocyte progenitor cells (MKPs).

In the present application, the term “platelet” generally refers to an acaryotic cytosome. Platelets may be formed by small pieces of cytoplasm that fall off during the lysis of megakaryocytic progenitor cells, and they play an important role in the physiological hemostasis process. They may be activated by thrombin, quickly adhere to the wound, and aggregate into clumps to form softer hemostatic plugs, then promote blood coagulation and form solid hemostatic plugs.

In the present application, that term “marker phenotype” generally refers to the identification of a marker or antigen on cells to determine their phenotype (e.g., differentiation status and/or cell type). For example, immunophenotyping can be used, which uses antibodies to recognize antigens presented on cells. Antibodies can be either monoclonal or polyclonal and those having minimal cross-reactivity with other cellular markers are usually selected. These markers that identify the same cell type among species can be identified based on the same markers, which may differ in structure (e.g., amino acid sequence) among species. Cell markers may include cell differentiation markers, and gene expression markers. Gene expression markers can include expressed genes that are indicative of the cell type or differentiation state.

In the present application, the term “proliferation” generally refers to the generation of multiple individual cells by the division of an initial cell. The multiple individual cells may be cells of the same type or cells of different types. The initial cells used for proliferation need not be the same as the cells resulting from the proliferation. For example, the proliferated cells may result from the growth and differentiation of a starting population of cells.

In the present application, the term “differentiation” generally refers to the process by which non-specific or less specific cells acquire the characteristic of particular cells. Differentiated or differentiation-induced cells are cells that have taken up a more specific position in a cell lineage.

In the present application, the term “maintain” generally refers to keeping the original state. In the present application, the term “maintain” means that during the culture process, the activity of cells remains unchanged at a certain level, and during the maintenance process, the cells can proliferate and/or differentiate.

In the present application, the term “composition” generally refers to products comprising specified amounts of specified ingredients, and any products produced directly or indirectly from a combination of specified amounts of specified ingredients. In the present application, the composition may further comprise other inactive ingredients, for example, carriers, excipients, adjuvants, and stabilizers.

In the present application, the term “ex vivo” generally refers to the manipulation of cells, tissues and/or organs that have been removed from the body of the organism. In some embodiments, the cells, tissues and/or organs may be returned to the organism by certain methods, or introduced into another organism.

In the present application, the term “in vitro” generally refers to the removal or release of a portion of an organism from the organism.

In the present application, the term “comprise”, “comprises”, and “comprising” generally means to including, generalizing, containing or encompassing. In some cases, it also means “being” and “consisting of”.

In the present application, the term “and/or” shall be understood to mean any one, two, or more of the alternatives, or any combination thereof.

In the present application, the term “about” typically refers to a change within a range of 0.5%-10% greater than or less than a specified value, e.g., a change within a range of 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% greater than or less than the specified value.

### DETAILED DESCRIPTION OF THE INVENTION

### Methods

In one aspect, the present application provides a method for obtaining platelets by differentiation, comprising adding a platelet lysate to a platelet differentiation culture medium. For example, the platelet lysate is a human platelet lysate.

In the present application, the initiating cells for platelet differentiation can be any cells with a platelet differentiation potential. For example, the initiating cells for platelet differentiation can be of natural origin or can be modified. For example, the initiating cells for platelet differentiation can be artificially modified by physical methods, chemical methods and/or biological methods. For example, the expression of certain genes of the initiating cells for platelet differentiation can be regulated. For example, the initiating cells for differentiation can be selected from one or more in the group of pluripotent cells, human induced pluripotent stem cells, human embryonic stem cells hematopoietic stem/progenitor cells, MKPs, etc.

In the present application, the source of the initiating cells for platelet differentiation is not limited, and can be of mammalian origin or non-mammalian origin. For example, the initiating cells for differentiation are differentiated from the pluripotent cells. For example, the initiating cells for differentiation are differentiated from the human embryonic stem cells. For example, the initiating cells for differentiation are differentiated from the human induced pluripotent stem cells. For example, the initiating cells for differentiation are differentiated from the hematopoietic stem/progenitor cells. For example, the initiating cells for differentiation are derived from human blood ex vivo. For example, the initiating cells for differentiation are derived from umbilical cord blood. For example, the initiating cells for differentiation are derived from bone marrow.

In the present application, the method comprises the process of proliferating pluripotent cells and/or differentiating them into platelets. In the present application, the method comprises the process of proliferating hematopoietic stem/progenitor cells and/or differentiating them into platelets. In the present application, the method comprises the process of proliferating MKPs and/or differentiating them into platelets. In the present application, the method comprises the process of proliferating human induced pluripotent cells and/or differentiating them into platelets. In the present application, the method comprises the process of proliferating human embryonic stem cells and/or differentiating them into platelets.

In the present application, the platelet differentiation culture medium used in the method can be a serum-free culture medium. For example, the platelet differentiation culture medium can comprise any platelet differentiation culture medium known in the art, and a human platelet lysate is added to the platelet differentiation culture medium to promote the differentiation of the differentiation-initiating cells into platelets.

In the present application, in the platelet differentiation culture medium, the human platelet lysate can be used to replace serum to promote the differentiation of the differentiation-initiating cells into platelets. For example, the serum can be fetal bovine serum. For example, the serum can be AB serum.

In the present application, the culture method may comprise the following steps: (1) inoculating hematopoietic stem/progenitor cells into the platelet differentiation culture medium I to obtain MKPs; (2) replacing the platelet differentiation culture medium I with the platelet differentiation culture medium II; (3) continuing to culture the cells using the platelet differentiation culture medium II with a complete medium replacement; (4) continuing to culture the MKPs in the platelet differentiation culture medium II supplemented with a metalloproteinase inhibitor to obtain platelets.

In the present application, the culture method may comprise the following steps: (1) inoculating hematopoietic stem/progenitor cells into the platelet differentiation culture medium I for about 7 days to obtain MKPs; (2) replacing the platelet differentiation culture medium I with the platelet differentiation culture medium II; (3) continuing to culture the cells for about 10 days using the platelet differentiation culture medium II with a complete medium replacement; (4) continuing to culture the MKPs in the platelet differentiation culture medium II supplemented with a metalloproteinase inhibitor for about 2 days to obtain platelets.

In the present application, the culture can be carried out under a culture condition of about 35-39° C. For example, about 34.5° C., about 35° C., about 35.5° C., about 36° C., about 36.5° C., about 37° C., about 37.5° C., about 38° C., about 38.5° C., about 39° C., or about 39.5° C.

In the present application, the culture can be carried out under a culture condition of about 3-7% CO₂. For example, about 3% CO₂, about 3.5% CO₂, about 4% CO₂, about 4.5% CO₂, about 5% CO₂, about 5.5% CO₂, about 6% CO₂, about 6.5% CO₂, about 7% CO₂, or about 7.5% CO₂.

In the present application, the culture method may be carried out under a culture condition with serum.

In the present application, the culture method may be carried out under a culture condition without serum.

In the present application, the culture method may be carried out under a culture condition without feeding.

In the present application, the culture method may be carried out under a culture condition with feeding.

In the present application, one or more substances can be added to the culture medium in the culture method, including but not limited to nutrients/extracts, growth factors, hormones, cytokines and/or culture medium supplements.

In the present application, the method may be an in vitro method.

In the present application, the method may be an ex vivo method.

In the present application, the method may be one not aimed at the diagnosis and treatment of diseases.

### Culture Medium

In another aspect, the present application provides a culture medium comprising the platelet differentiation culture medium supplemented with a human platelet lysate (hPL).

In the present application, the method can comprise using the culture medium described in the present application for differentiation and/or proliferation and/or maintenance of cells to obtain platelets.

In the present application, the platelet differentiation culture medium can comprise no serum.

In the present application, the platelet differentiation culture medium can comprise a basal culture medium. For example, the basal culture medium can comprise any culture medium known in the art. For example, the basal culture medium can comprise one or more of IMDM, MEM, Ham's F12, mTeSR1, APEL, StemSpan™ SFEM II, DMEM, and RPMI1640. For example, the basal culture medium comprises one or more of StemSpan™ SFEM II and IMDM. For example, the basal culture medium is StemSpan™ SFEM II. For example, the basal culture medium is IMDM.

In the present application, the platelet differentiation culture medium can comprise one or more of nutrients, extracts, growth factors, hormones, cytokines, and culture medium supplements.

For example, one or more of the following substances can be added to the platelet differentiation culture medium, including but not limited to serum alternative, glutamine, NEAAs (non-essential amino acids), Ascorbic acid, epidermal growth factors (EGFs), acidic fibroblast growth factor (aFGF), basic fibroblast growth factor (bFGF), leukemia inhibitory factor (LIF), hepatocyte growth factor (HGF), insulin-like growth factor-1 (IGF-1), insulin-like growth factor-2 (IGF-2), keratinocyte growth factor (KGF), nerve growth factor (NGF), platelet-derived growth factor (PDGF), transforming growth factor-β (TGF-β), bone morphogenetic protein (BMP4), vascular endothelial growth factor (VEGF), transferrin, insulin, selenium, various interleukins (e.g., IL-1 to IL-18), various colony stimulating factors (e.g., granulocyte/macrophage colony stimulating factor (GM-CSF)), various interferons (e.g., IFN-γ), stem cell factor (SCF), thrombopoietin (TPO), erythropoietin (EPO), N2 supplement, B27 supplement, and Fms-related tyrosine kinase 3 ligand (FLt3L). The added substances can be from any source and can be commercially available, natural or recombinant.

For example, inhibitors can be added to the platelet differentiation culture medium, and the inhibitors can include, but are not limited to GSK-3 inhibitors, MEK inhibitors, ROCK inhibitors, metalloproteinase inhibitors, and the like.

For example, the ROCK inhibitors include, but are not limited to polynucleotides, polypeptides and small molecules, which can reduce ROCK expression and/or ROCK activity. For example, the ROCK inhibitors can be Y27632, Thiazovivin, Fasudil (HA-1077), GSK429286A, RKI-1447, WAY-624704, H-1152, Azaindole 1 (TC-S 7001), Hydroxyfasudil (HA-1100), Y-39983, Netarsudil (AR-13324), GSK269962A, Ripasudil (K-115) hydrochloride dihydrate, Belumosudil (KD025), AT13148, Emetine hydrochloride, ZINC00881524 or an antibody against the ROCK pathway.

For example, the metalloproteinase inhibitors can be GM6001, CL-82198, SM-7368, Dendrobii caulis Extract, T-26c, AUDA (compound 43), MMP-9-IN-1 (OUN87710), Isoliquiritin apioside (ISLA, ILA), Nobiletin (NSC 76751, Hexamethoxyflavone), Solasodine, Triolein, Cordycepin (3′-Deoxyadenosine), Doxycycline Hyclate (WC2031), Trans-Zeatina ((E)-Zeatin), Lactobionic acid (Galactosylgluconic acid), Doxycycline, Morroniside, 1,10-Phenanthroline, JNJ0966, Abametapir (HA-44, BRN 0123183), o-Phenanthroline, Batimastat (BB-94), Marimastat (BB-2516, TA2516), SB-3CT, TAPI-1, NSC 405020, T-5224, Auraptene (7-geranyloxycoumarin), Polygalacic acid, ARP 100, Absinthin (Absynthine), or Ginkgolide C (BN-52022).

For example, the platelet differentiation culture medium in the culture medium further comprises one or more of IL-3, IL-6, SCF, TPO, FLt3L, N2 supplement, B27 supplement, NEAAs, Glutamax, ITS, and Ascorbic acid.

For example, the platelet differentiation culture medium in the culture medium further comprises IL-3, IL-6, SCF, TPO, and FLt3L.

For example, the platelet differentiation culture medium in the culture medium further comprises IL-6, SCF, TPO, N2 supplement, B27 supplement, NEAAs, Glutamax, ITS, and Ascorbic acid.

For example, the platelet differentiation culture medium in the culture medium further comprises platelet differentiation culture medium I, wherein the platelet differentiation culture medium I comprises StemSpan™ SFEM II, IL-3, IL-6, SCF, TPO, FLt3L, and hPL.

For example, the platelet differentiation culture medium in the culture medium further comprises platelet differentiation culture medium II, wherein the platelet differentiation culture medium II comprises IMDM, N2 supplement, B27 supplement, NEAAs, Glutamax, ITS, Ascorbic acid, IL-6, SCF, TPO, and hPL.

For example, in the platelet differentiation culture medium, the concentration of the IL-3 is about 5-100 ng/ml. For example, about 5 ng/ml, about 10 ng/ml, about 15 ng/ml, about 20 ng/ml, about 25 ng/mL, about 30 ng/ml, about 35 ng/ml, about 40 ng/ml, about 45 ng/ml, about 50 ng/mL, about 55 ng/ml, about 60 ng/mL, about 65 ng/ml, about 70 ng/ml, about 75 ng/ml, about 80 ng/ml, about 85 ng/mL, about 90 ng/ml, about 95 ng/ml, about 100 ng/mL.

For example, in the platelet differentiation culture medium, the concentration of the IL-6 is about 5-100 ng/ml. For example, about 5 ng/ml, about 10 ng/mL, about 15 ng/mL, about 20 ng/ml, about 25 ng/mL, about 30 ng/ml, about 35 ng/ml, about 40 ng/ml, about 45 ng/ml, about 50 ng/mL, about 55 ng/ml, about 60 ng/ml, about 65 ng/ml, about 70 ng/ml, about 75 ng/ml, about 80 ng/ml, about 85 ng/ml, about 90 ng/mL, about 95 ng/ml, about 100 ng/mL.

For example, in the platelet differentiation culture medium, the concentration of the SCF is about 5-100 ng/ml. For example, about 5 ng/mL, about 10 ng/ml, about 15 ng/ml, about 20 ng/mL, about 25 ng/mL, about 30 ng/mL, about 35 ng/mL, about 40 ng/mL, about 45 ng/ml, about 50 ng/mL, about 55 ng/mL, about 60 ng/mL, about 65 ng/ml, about 70 ng/mL, about 75 ng/ml, about 80 ng/mL, about 85 ng/ml, about 90 ng/mL, about 95 ng/ml, about 100 ng/mL.

For example, in the platelet differentiation culture medium, the concentration of the TPO is about 5-100 ng/ml. For example, about 5 ng/ml, about 10 ng/mL, about 15 ng/ml, about 20 ng/mL, about 25 ng/mL, about 30 ng/mL, about 35 ng/mL, about 40 ng/ml, about 45 ng/ml, about 50 ng/mL, about 55 ng/mL, about 60 ng/mL, about 65 ng/ml, about 70 ng/mL, about 75 ng/ml, about 80 ng/mL, about 85 ng/mL, about 90 ng/mL, about 95 ng/mL, about 100 ng/mL.

For example, in the platelet differentiation culture medium, the concentration of the FLt3L is about 5-100 ng/ml. For example, about 5 ng/ml, about 10 ng/ml, about 15 ng/ml, about 20 ng/ml, about 25 ng/ml, about 30 ng/mL, about 35 ng/mL, about 40 ng/ml, about 45 ng/ml, about 50 ng/mL, about 55 ng/mL, about 60 ng/mL, about 65 ng/ml, about 70 ng/ml, about 75 ng/ml, about 80 ng/ml, about 85 ng/ml, about 90 ng/ml, about 95 ng/ml, about 100 ng/mL.

For example, in the platelet differentiation culture medium, the concentration of the N2 supplement is about 0.25%-1%. For example, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%.

For example, in the platelet differentiation culture medium, the concentration of the B27 supplement is about 0.5%-2%. For example, about 0.25%, about 0.3%, about 0.35%, about 0.40%, about 0.45%, about 0.5%, about 0.55%, about 0.6%, about 0.65%, about 0.7%, about 0.75%, about 0.8%, about 0.85%, about 0.9%, about 0.95%, about 1%, about 1.05%, about 1.1%, about 1.15%, about 1.2%, about 1.25%, about 1.3%, about 1.35%, about 1.4%, about 1.45%, about 1.5%, about 1.55%, about 1.6%, about 1.65%, about 1.7%, about 1.75%, about 1.8%, about 1.85%, about 1.9%, about 1.95%, about 2%.

For example, in the platelet differentiation culture medium, the concentration of the Glutamax is about 0.5%-2%. For example, about 0.25%, about 0.3%, about 0.35%, about 0.40%, about 0.45%, about 0.5%, about 0.55%, about 0.6%, about 0.65%, about 0.7%, about 0.75%, about 0.8%, about 0.85%, about 0.9%, about 0.95%, about 1%, about 1.05%, about 1.1%, about 1.15%, about 1.2%, about 1.25%, about 1.3%, about 1.35%, about 1.4%, about 1.45%, about 1.5%, about 1.55%, about 1.6%, about 1.65%, about 1.7%, about 1.75%, about 1.8%, about 1.85%, about 1.9%, about 1.95%, about 2%.

For example, in the platelet differentiation culture medium, the concentration of the ITS is about 0.5%-2%. For example, about 0.25%, about 0.3%, about 0.35%, about 0.40%, about 0.45%, about 0.5%, about 0.55%, about 0.6%, about 0.65%, about 0.7%, about 0.75%, about 0.8%, about 0.85%, about 0.9%, about 0.95%, about 1%, about 1.05%, about 1.1%, about 1.15%, about 1.2%, about 1.25%, about 1.3%, about 1.35%, about 1.4%, about 1.45%, about 1.5%, about 1.55%, about 1.6%, about 1.65%, about 1.7%, about 1.75%, about 1.8%, about 1.85%, about 1.9%, about 1.95%, about 2%.

For example, in the platelet differentiation culture medium, the concentration of the Ascorbic acid is about 50-200 μM. For example, about 50 μM, about 55 μM, about 60 μM, about 65 μM, about 70 μM, about 75 μM, about 80 μM, about 85 μM, about 90 μM, about 95 μM, about 100 μM, about 105 μM, about 110 μM, about 115 μM, about 120 μM, about 125 μM, about 130 μM, about 135 μM, about 140 μM, about 145 μM, about 150 μM, about 155 μM, about 160 μM, about 165 μM, about 170 μM, about 175 μM, about 180 μM, about 185 μM, about 190 μM, about 195 μM, about 200 μM.

For example, in the platelet differentiation culture medium, the concentration of the hPL is about 0.5%-10%. For example, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%.

In the present application, the platelet differentiation culture medium can be used alone or in combination with other culture media. For example, different differentiation culture media can be used for different differentiation steps.

### Cells

In another aspect, the present application further provides a platelet which can be prepared by the method described in the present application or can be obtained by culturing using the culture medium described in the present application.

In the present application, the cells and/or the state of the cells can be determined by cell markers. For example, the type of cells and/or the state of cells can be determined by marker phenotype.

In some embodiments, the cells according to the present application are isolated.

### Composition and Use

In another aspect, the present application further provides a composition comprising pluripotent cells and the culture medium.

In another aspect, the present application further provides a composition comprising hematopoietic stem/progenitor cells and the culture medium.

In another aspect, the present application further provides a composition comprising MKPs and the culture medium.

In another aspect, the present application further provides a culture platform for obtaining platelets, the culture platform comprises the method and the culture medium.

In another aspect, the present application further provides a method for preventing and/or treating diseases, the method comprises administering the platelets to a subject in need thereof, wherein the platelets are obtained by using the method, the culture medium, the composition or the culture platform.

For example, the platelets administered can be pharmaceutically formulated according to any conventional method. For example, the active ingredients can be mixed or diluted with carriers, excipients or diluents. Examples of suitable carriers, excipients or diluents are lactose, dextrose, sucrose, sorbitol, mannitol, glycine, polyethylene glycol, starch, gum arabic, alginic acid, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil. The preparation may additionally comprise, for example, fillers, anticoagulants, lubricants, wetting agents, flavoring agents, emulsifiers, preservatives, and the like. The compositions of the invention may be formulated by using any methods well known in the art so as to provide fast, sustained or delayed release of the active ingredients after administered to patients.

The cells in the present application can be administered by injection (e.g., intramuscularly, intravenously, intraperitoneally, subcutaneously), or by other methods such as infusion to ensure that they enter the bloodstream in an effective form. The cells may also be administered via intratumoral, peritumoral, intralesional or perilesional routes to exert local and systemic therapeutic effects. For example, they may be administered topically or intravenously.

In the present application, the administration dose of the cells may also be a single dose or multiple doses. For example, the actual amount of cells administered may be determined based on a variety of relevant factors, such as the type of disease; the route of administration; the age, sex and/or weight of the patient; and the severity of the patient's symptoms.

Without wishing to be bound by any theory, the following examples are only for illustrating the various technical solutions of the present application, and are not used to limit the scope of the invention of the present application.

### EXAMPLES

### Example 1. Differentiation and Culture of Platelets

The production of platelets was induced by using the following methods and culture media:
(1) The hematopoietic stem/progenitor cell suspension was seeded into a 6-well suspension culture plate (8×10⁴ cells per well), and was cultured under conditions of 37° C. and 5% CO₂ for 7 days (recorded as Day 1 to Day 7) in the platelet differentiation culture medium I;
(2) Half of the medium was replaced every 3 days with fresh platelet differentiation culture medium I;
(3) Megakaryocyte progenitor cells (MKPs) could be obtained on Day 7;
(4) The MKPs were collected, centrifuged at 400 g for 10 min, the supernatant was discarded, and the cells were resuspended in the platelet differentiation culture medium II, and seeded into a 6-well suspension culture plate (8× 10⁵ cells per well). The cells were cultured under conditions of 37° C. and 5% CO₂ for 5 days, i.e., Day 7 to Day 12;
(5) Fresh platelet differentiation culture medium II was supplemented once on Day 10;
(6) On Day 12, fresh platelet differentiation culture medium II was supplemented once, and the cells were transferred to conditions of 39° C. and 5% CO₂, and continuously cultured in the differentiation culture medium II for another 5 days, i.e., Day 12 to Day 17;
(7) Two days before harvesting, i.e., on Day 15, GM6001 was added to each well, and the cells were cultured for another 2 days under conditions of 39° C. and 5% CO₂;
(8) On Day 17, after the cells were harvested, they were centrifuged at 1000 g for 10 min to obtain a high-purity platelet suspension;
(9) The platelet differentiation culture medium I: StemSpan™ SFEM II, IL-3, IL-6, SCF, TPO, FLt3L, and hPL;

The platelet differentiation culture medium II: IMDM, N2 supplement, B27 supplement, NEAAs, Glutamax, ITS, Ascorbic acid, IL-6, SCF, TPO, and hPL.

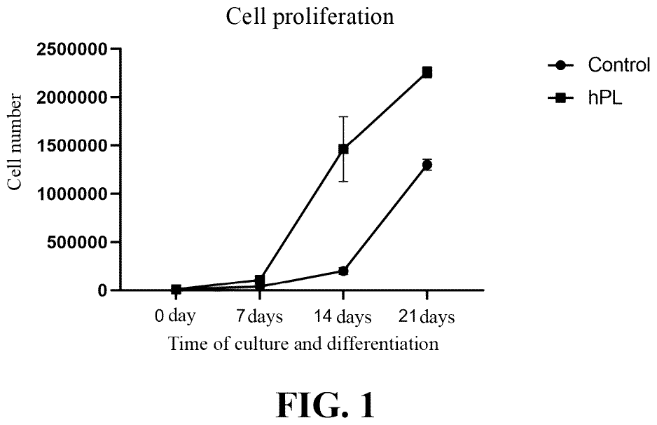

The experimental results are shown in FIG. 1. Under the same culture conditions, the number of cultured cells was detected on Day 0, Day 7, Day 14 and Day 21, respectively. Compared with the control group (hPL was not added to the culture medium), the hPL group significantly promoted cell proliferation for 7-21 days after culture. The above experimental results demonstrate that adding the human platelet lysate (hPL) to the culture medium could promote the proliferation of hematopoietic stem/progenitor cells and/or their differentiation into platelets.

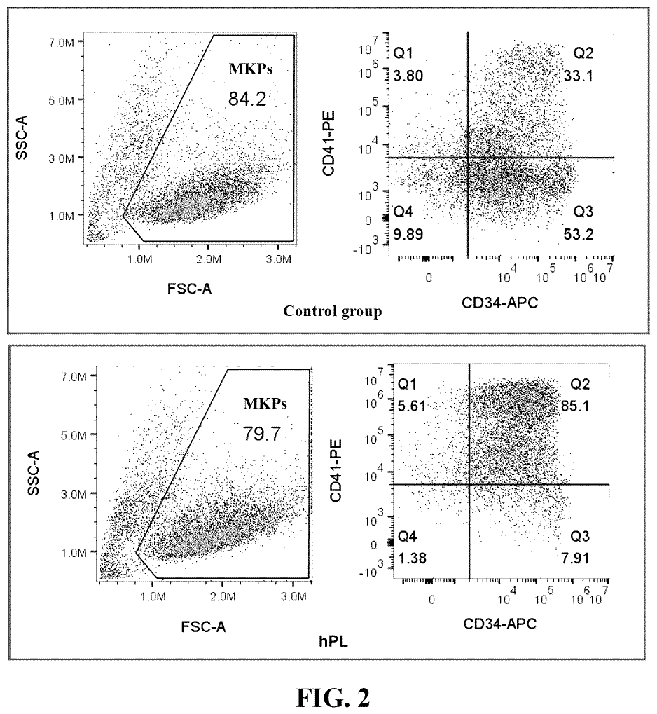

The experimental results are shown in FIG. 2. Under the same culture conditions, the cultured cells were detected on Day 7. Compared with the control group (hPL was not added to the culture medium), the hPL group could better promote the cells to produce more precursor cells (MKPs). The above experimental results demonstrate that adding the human platelet lysate (hPL) to the culture medium could promote the proliferation of hematopoietic stem/progenitor cells and/or their differentiation into the precursor cells (MKPs), and then the proliferation of megakaryocyte progenitor cells (MKPs) and/or their differentiation into platelets, thereby promoting the proliferation of hematopoietic stem/progenitor cells and/or their differentiation into platelets.

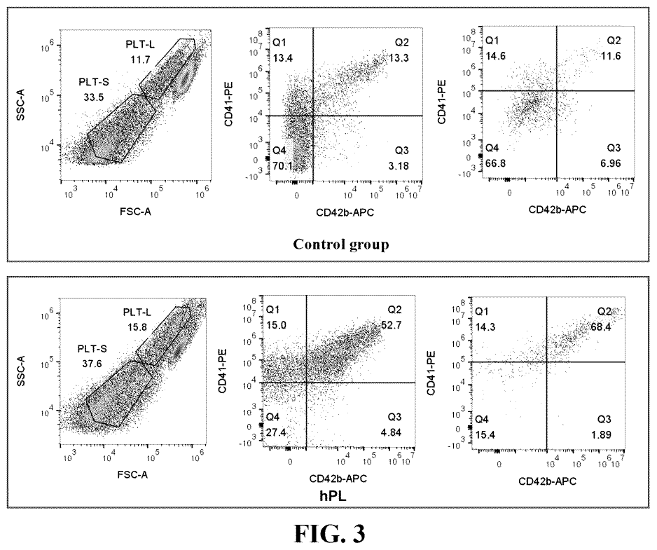

The experimental results are shown in FIG. 3. Under the same culture conditions, the number of cultured cells was detected on Day 17. Compared with the control group (hPL was not added to the culture medium), the hPL group could better promote the cells to produce more platelets (PLTs). The above experimental results demonstrate that adding the human platelet lysate (hPL) to the culture medium could promote the proliferation of hematopoietic stem/progenitor cells and/or their differentiation into platelets.

1. A method for inducing the proliferation and/or differentiation of pluripotent cells, hemopoietic stem/progenitor cells or megakaryocytic progenitor cells (MKPs) into platelets, comprising adding human platelet lysate (hPL) to a platelet differentiation culture medium.

2. The method of claim 1, wherein said platelet differentiation culture medium comprises hPL, IL-3, IL-6, SCE, TPO, and FLt3L.

3. The method of claim 1, wherein said platelet differentiation culture medium comprises hPL, IL-6, SCF, TPO, N2 supplement, B27 supplement, NEAAs, Glutamax, ITS, and ascorbic acid.

4. (canceled)

5. The method of claim 1, wherein said pluripotent cells are human embryonic stem cells or human induced pluripotent stem cells; said hematopoietic stem/progenitor cells are CD34⁺ hematopoietic stem/progenitor cells.

6-20. (canceled)

21. The method of claim 1, wherein said platelet differentiation culture medium comprises a basal culture medium, wherein id basal culture medium is selected from one or more in the group of IMDM, MEM, Ham's F12, mTeSR1, APEL, Stem Span™ SFEM II, DMEM, and RPMI1640.

22-25. (canceled)

26. The method of claim 1, wherein said platelet differentiation culture medium further comprises nutrients, extracts, growth factors, hormones, cytokines, and culture medium supplements.

27. The method of claim 1, wherein said platelet differentiation culture medium further comprises one or more of IL-3, IL-6, SCF (stem cell factor), TPO (thrombopoietin), FLt3L (Fms-related tyrosine kinase 3 ligand), N2 supplement, B27 supplement, NEAAs (non-essential amino acids), Glutamax (glutamine), ITS (insulin, transferrin, selenium), and Ascorbic acid.

28. (canceled)

29. (canceled)

30. The method of claim 2, wherein the concentration of said hPL is about 0.5%-10%, the concentration of said IL-3 is about 5-100 ng/ml, the concentration of said IL-6 is about 5-100 ng/ml, the concentration of said SCF is about 5-100 ng/ml, the concentration of said TPO is about 5-100 ng/ml and the concentration of said FLt3L is about 5-100 ng/ml.

31-34. (canceled)

35. The method of claim 3, wherein the concentration of said hPL is about 0.5%-10%, the concentration of said N2 supplement is about 0.25%-1%, the concentration of said B27 supplement is about 0.5%-2%, the concentration of said Glutamax is about 0.5%-2%, the concentration of said ITS is about 0.5%-2%, the concentration of said ascorbic acid is about 50-200 μM, the concentration of said IL-6 is about 5-100 ng/ml, the concentration of said SCF is about 5-100 ng/mL, and the concentration of said TPO is about 5-100 ng/mL.

36-40. (canceled)

41. The method of claim 1, wherein said platelet differentiation culture medium comprises platelet differentiation culture medium I, said platelet differentiation culture medium I comprises StemSpan™ SFEM II, IL-3, IL-6, SCF, TPO, FLt3L, and hPL.

42. The method of claim 1, wherein said platelet differentiation culture medium comprises platelet differentiation culture medium II, said platelet differentiation culture medium II comprises IMDM, N2 supplement, B27 supplement, NEAAs, Glutamax, ITS, Ascorbic acid, IL-6, SCF, TPO, and hPL.

43. The method of claim 41, wherein said method comprises culturing the hematopoietic stem/progenitor cells in the platelet differentiation culture medium I for about 7 days.

44. The method of claim 42, wherein said method comprises culturing the MKPs in the platelet differentiation culture medium II for about 10 days.

45. The method of claim 44, wherein said method comprises continuously culturing the MKPs in the platelet differentiation culture medium II supplemented with a metalloproteinase inhibitor for about 2 days.

46. The method of claim 45, wherein said metalloproteinase inhibitor is GM6001.

47. The method of claim 44, wherein said method comprises following steps: (1) culturing the hematopoietic stem/progenitor cells in the platelet differentiation culture medium I for about 7 days, (2) culturing the MKPs in the platelet differentiation culture medium II for about 10 days, and (3) culturing the MKPs in the platelet differentiation culture medium II supplemented with a metalloproteinase inhibitor for about 2 days.

48-70. (canceled)

71. A culture medium to culturing platelets, wherein said culture medium comprises platelet differentiation culture medium I, said platelet differentiation culture medium I comprises StemSpan™ SFEM II, IL-3, IL-6, SCF, TPO, FLt3L, and hPL.

72. The culture medium of claim 71, wherein said culture medium further comprises platelet differentiation culture medium II, said platelet differentiation culture medium II comprises IMDM, N2 supplement, B27 supplement, NEAAs, Glutamax, ITS, Ascorbic acid, IL-6, SCF, TPO, and hPL.

73-76. (canceled)

77. A composition comprising cells and the culture medium of claim 71, wherein said cells are selecting from the groups consisting of pluripotent cells, hematopoietic stem/progenitor cells, and MKPs.

78. (canceled)

79. (canceled)

80. A culture platform for obtaining platelets, comprising the method of claim 1.

81-84. (canceled)
